(19) United States
(12) Patent Application Publication
(10) Pub. No.: US 2008/0234363 A1
(43) Pub. Date: Sep. 25, 2008

(54) Antitumoral Compounds

(75) Inventors: Jose Fernando Reyes Benitez, Madrid (ES); Jose Antonio Jimenez Guerrero, Madrid (ES); Andres Manuel Francesch Solloso, Madrid (ES); Maria Del Carmen Cuev As Marchante, Madrid (ES)

Correspondence Address: KING & SPALDING, 1185 AVENUE OF THE AMERICAS, NEW YORK, NY, 10036-4003, US

(73) Assignee: PHARMA MAR, S.A., Madrid (ES)

(21) Appl. No.: 11/996,992

(22) PCT Filed: Aug. 1, 2006
(86) PCT No.: PCT/GB06/50229

§ 371 Date: Mar. 17, 2008

(30) Foreign Application Priority Data
Aug. 1, 2005 (GB) 0515673.2

(51) Int. Cl. A61K 31/352; C07D 311/02; A61P 35/00

(52) U.S. Cl. 514/456; 549/401

(57) ABSTRACT

Compounds of general formula (I), wherein R₁, R₂, R₃, R₄, R₅, R₆ and Y are as defined, and X group is O, S(O)ₘ or NR; are of use in the treatment of cancer.

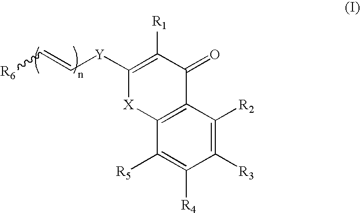

### FIELD OF THE INVENTION

The present invention relates to new antitumoral compounds, pharmaceutical compositions containing them and their use as antitumoral agents.

### BACKGROUND OF THE INVENTION

Cancer is a leading cause of death in animals and humans. Huge efforts have been and are still being undertaken in order to obtain an antitumor agent active and safe to be administered to patients suffering from a cancer. The problem to be solved by the present invention is to provide compounds that are useful in the treatment of cancer.

### SUMMARY OF THE INVENTION

In one aspect, the present invention is directed to antitumor compounds of general formula I or a pharmaceutically acceptable salt, derivative, prodrug or stereoisomer thereof

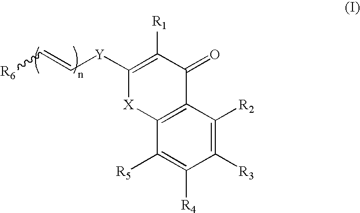

wherein R₁, R₂, R₃, R₄, R₅ and R₆ are each independently selected from the group consisting of hydrogen, ORₐ, OC(═O)Rₐ, halogen, substituted or unsubstituted C₁-C₁₂ alkyl, substituted or unsubstituted C₂-C₁₂ alkenyl and substituted or unsubstituted C₂-C₁₂ alkynyl;
wherein Rₐ is selected from the group consisting of hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl and substituted or unsubstituted heterocyclic group;
wherein X is O, S(O)ₘ or NR; wherein m is 0, 1 or 2;
wherein R is selected from the group consisting of hydrogen, substituted or unsubstituted C₁-C₁₂ alkyl, substituted or unsubstituted C₂-C₁₂ alkenyl and substituted or unsubstituted C₂-C₁₂ alkynyl;
wherein Y represents a substituted or unsubstituted C₁-C₁₂ alkylene chain;
wherein n is from 2 to 6;
and the wavy line ( [symbol] ) means that the bond can exist as (E)-isomer or (Z)-isomer.

Some of these compounds are known compounds.

Stigmatellin A, was isolated from Stigmatella aurantiaca by B. Kunze et al (J. Antibiot. (1984), 37, 454-61):

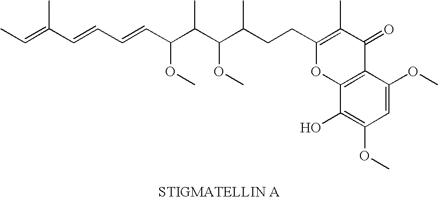

It is disclosed that this compound blocks the electron flow in the respiratory chain of bovine heart submitochondrial particles at the site of the cytochrome b-cl segment, giving rise an antibiotic activity. Its inhibitory potency was identical with that of antimycin and myxothiazol, and like these antibiotics, stigmatellin A caused a shift in the spectrum of reduced cytochrome b. (G. Thierbach et al. Biochimica et Biophysica Acta (1984), 765, 227-35). This article also describes the inhibitory activity and the structure of some stigmatellin derivatives, for example the following derivative is described:

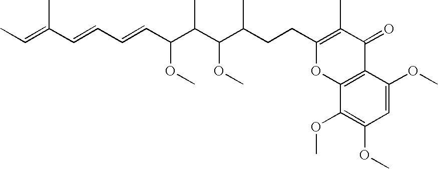

It is remarkable that none of the derivatives described was more efficient than the natural compound produced by the myxobacterium.

There are others stigmatellin derivatives described in the prior art, see for example:

G. Hoefle et al. “Antibiotics from gliding bacteria, XXIII. Stigmatellin A and B—two novel antibiotics from Stigmatella aurantiaca (Myxobacterales)” Liebigs Annalen der Chemie (1984), 12, 1883-1904, which in addition to Stigmatellin A and B also describe the activity and the structure of the following synthetic stigmatellin derivatives:

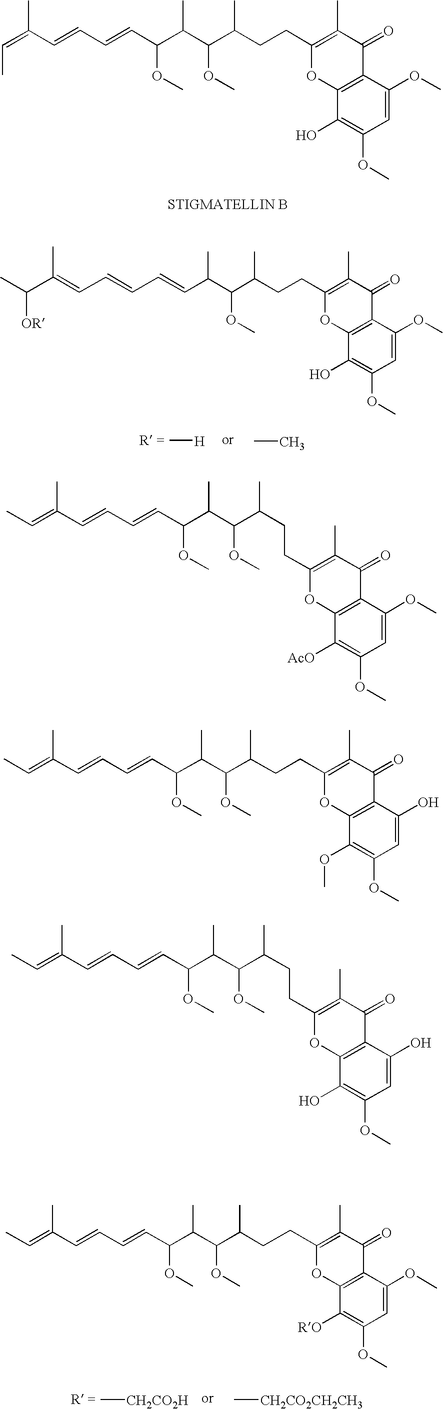

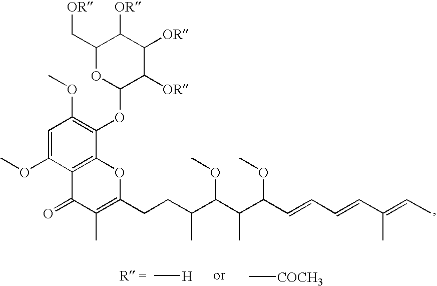

N. Gaitatzis et al. “The Biosynthesis of the Aromatic Myxobacterial Electron Transport Inhibitor Stigmatellin Is Directed by a Novel Type of Modular Polyketide Synthase” Journal of Biological Chemistry (2002), 277, 13082-13090, which in addition to the biosynthesis of Stigmatellin also describes the structures of Stigmatellins X and Y, their activity as inhibitors of Myxobacterial electron transport and the antifungal activity of Stigmatellin Y.

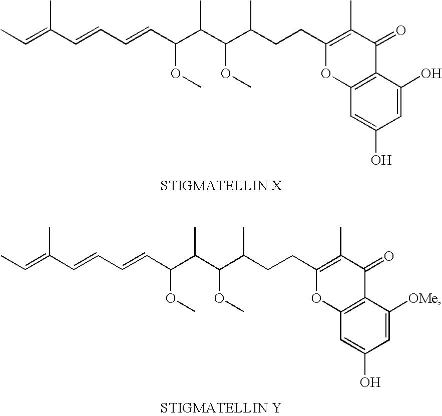

L. Domon and D. Uguen, “Toward a total synthesis of stigmatellin; obtention of an advanced fragment from gallic acid” Tetrahedron Letters (2000), 41, 5501-5505, which describes one O-benzyl stigmatellin.

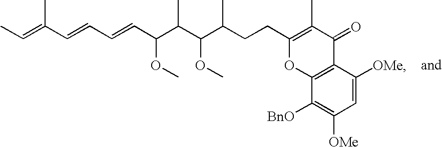

K. M. Giangiacomo et al. “Stigmatellin and other electron transfer inhibitors as probes for the Q_{b} binding site in the reaction center of photosynthetic bacteria” Prog. Photosynth. Res., Proc. Int. Congr. Photosynth., 7th (1987), Meeting Date 1986, 2 409-12, which in addition to the use of Stigmatellin as a probe for the Q_{b} binding site also describes the activity and the structure of Stigmatellin II.

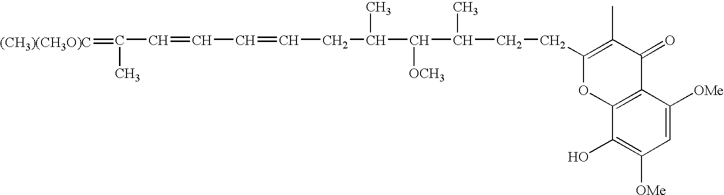

The natural Stigmatellins A and B showed better antibiotic activity than the above mentioned synthetic compounds.

Stigmatellin A is also disclosed as a powerful inhibitor of photosynthetic electron transport. (“Stigmatellin. A dual type inhibitor of photosynthetic electron transport”, O. Walter et al. Biochimica et Biophysica Acta (1985), 807, 216-19.)

There is no disclosure in the prior art of antitumor activity for Stigmatellin A, B and their derivatives.

We make no claim to the known compounds. Specifically, we make no claim to the known compounds disclosed in the literature cited above. Accordingly, in respect of our claim to the compounds per se, we have constructed the proviso such that:

(a) when the structure is:

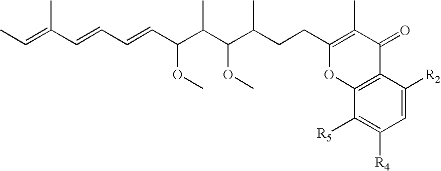

and R₂ is —OCH₃, R₄ is —OCH₃; R₅ is not —OH, —OCH₃, —OCOCH₃, —OCH₂CO₂H, —OCH₂Ph or —OCH₂CO₂CH₂CH₃;
when R₂ is —OH, R₄ is OCH₃; R₅ is not —OH or —OCH₃;
and
when R₄ is —OH, R₅ is —H; R₂ is not —OH or —OCH₃;
(b) when the structure is:

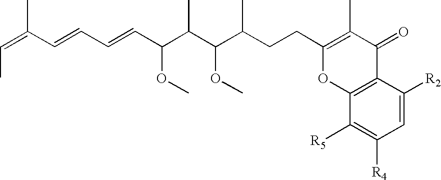

### and R₂ is —OCH₃; R₄ is —OCH₃; R₅ is not —OH;

(c) when the structure is:

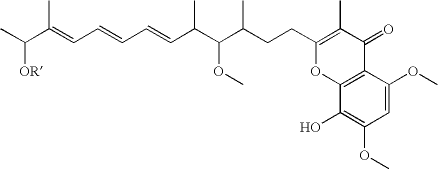

R′ is not H or methyl;
(d) when the structure is:

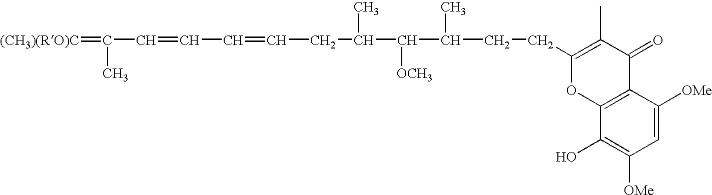

R′ is not methyl; and
(e) when the structure is:

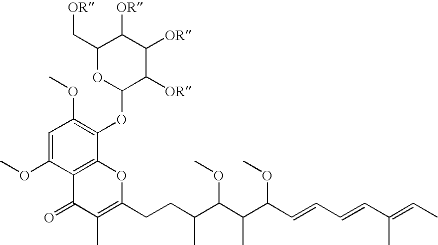

the R″ groups are not all —H or are not all —COCH₃.

In another aspect, the present invention is directed to pharmaceutical compositions comprising a compound of formula I, as defined above, or pharmaceutically acceptable salts, derivatives, prodrugs or stereoisomers thereof together with a pharmaceutically acceptable carrier or diluent.

In another aspect, the present invention is also directed to the use of compounds of formula I

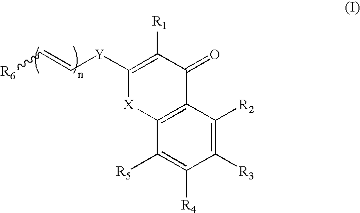

wherein R₁, R₂, R₃, R₄, R₅ and R₆ are each independently selected from the group consisting of hydrogen, ORₐ, OC(═O)Rₐ, halogen, substituted or unsubstituted C₁-C₁₂ alkyl, substituted or unsubstituted C₂-C₁₂ alkenyl and substituted or unsubstituted C₂-C₁₂ alkynyl;
wherein Rₐ is selected from the group consisting of hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl and substituted or unsubstituted heterocyclic group;
wherein X is O, S(O)ₘ or NR; wherein m is 0, 1 or 2;
wherein R is selected from the group consisting of hydrogen, substituted or unsubstituted C₁-C₁₂ alkyl, substituted or unsubstituted C₂-C₁₂ alkenyl and substituted or unsubstituted C₂-C₁₂ alkynyl;
wherein Y represents a substituted or unsubstituted C₁-C₁₂ alkylene chain;
wherein n is from 0 to 6; and the wavy line ( [symbol] ) means that the bond can exist as (E)-isomer or (Z)-isomer, when n≧1;
or pharmaceutically acceptable salts, derivatives, prodrugs or stereoisomers thereof in the treatment of cancer, or in the preparation of a medicament for the treatment of cancer.

Other aspects of the invention are methods of treatment, and compounds for use in the methods.

The present invention also relates to the isolation of the compounds of formula I from a porifera of the family Plakinidae genus Corticium sp., and the formation of derivatives from these compounds.

### DETAILED DESCRIPTION OF PREFERRED EMBODIMENTS

The present invention relates to compounds of general formula I as defined above.

In these compounds the substituents can be selected in accordance with the following guidance:

Alkyl and alkoxy groups may be branched or unbranched and preferably have from 1 to 12 carbon atoms. One more preferred class of alkyl and alkoxy groups has from 1 to about 6 carbon atoms. Methyl, ethyl, propyl, butyl and pentyl including isopropyl, isobutyl, isopentyl, methylbutyl and methylpentyl are particularly preferred alkyl groups in the compounds of the present invention. Methoxy, ethoxy, propoxy including isopropoxy are particularly preferred alkoxy groups in the compounds of the present invention.

Alkylene group refers to a straight or branched chain, divalent, saturated hydrocarbon group, preferably having from 1 to 12 carbon atoms. One more preferred class of alkylene groups has from 3 to about 8 carbon atoms. 1,3-Propylene, 1,4-butylene, 1,5-pentylene, 1,6-hexylene and 1,7-heptylene are particularly preferred alkylene groups in the compounds of the present invention.

Preferred alkenyl and alkynyl groups in the compounds of the present invention have one or more unsaturated linkages and from 2 to about 12 carbon atoms. One more preferred class of alkenyl groups has from 2 to about 6 carbon atoms, and most preferably 4 to 6 carbon atoms. One more preferred class alkynyl groups has from 2 to about 6 carbon atoms, and most preferably 2 to 4 carbon atoms.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Specially preferred aryl groups include substituted or unsubstituted phenyl, naphthyl, biphenyl, phenanthryl and anthracyl.

Suitable heterocyclic groups include heteroaromatic and heteroalicyclic groups. Suitable heteroaromatic groups in the compounds of the present invention contain one, two or three heteroatoms selected from N, O or S atoms and include, e.g., coumarinyl including 8-coumarinyl, quinolinyl including 8-quinolinyl, pyridyl, pyrazinyl, pyrimidyl, furyl, pyrrolyl, thienyl, thiazolyl, oxazolyl, imidazolyl, indolyl, benzofuranyl and benzothiazol groups. Suitable heteroalicyclic groups in the compounds of the present invention contain one, two or three heteroatoms selected from N, O or S atoms and include, e.g., tetrahydrofuranyl, tetrahydropyranyl, piperidinyl, morpholino and pyrrolidinyl groups.

The groups above mentioned may be substituted at one or more available positions by one or more suitable groups such as OR″, ═O (oxo group), SR″, SOR″, SO₂R″, NO₂, NHR″, N(R″)₂, ═N—R′, NHCOR″, N(COR″)₂, NHSO₂R″, CN, halogen, C(═O)R″, CO₂R″, OC(═O)R″ wherein each of the R′ groups is independently selected from the group consisting of H, OH, NO₂, NH₂, SH, CN, halogen, C(═O)H, C(═O)alkyl, CO₂H, substituted or unsubstituted C₁-C₁₂ alkyl, substituted or unsubstituted C₂-C₁₂ alkenyl, substituted or unsubstituted C₂-C₁₂ alkynyl and substituted or unsubstituted aryl. Suitable halogen substituents in the compounds of the present invention include F, Cl, Br and I. Where such groups are themselves substituted, the substituents may be chosen from the foregoing list.

The term “pharmaceutically acceptable salts, derivatives, prodrugs” refers to any pharmaceutically acceptable salt, ester, solvate, hydrate or any other compound which, upon administration to the recipient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The compounds of the invention may be in crystalline form either as free compounds or as solvates (e.g. hydrates) and it is intended that both forms are within the scope of the present invention. Methods of salvation are generally known within the art.

Any compound that is a prodrug of a compound of formula I is within the scope and spirit of the invention. The term “prodrug” is used in its broadest sense and encompasses those derivatives that are converted in vivo to the compounds of the invention. Such derivatives would readily occur to those skilled in the art, and include, for example, compounds where a free hydroxy group is converted into an ester derivative.

The compounds of the present invention represented by the above described formula I may include enantiomers depending on heir asymmetry or diastereoisomers. Stereoisomerism about the double bond is also possible, therefore in some cases the molecule could exist as (E)-isomer or (Z)-isomer. The single isomers and mixtures of the isomers fall within the scope of the present invention.

Preferred compounds of the invention are those of general formula I

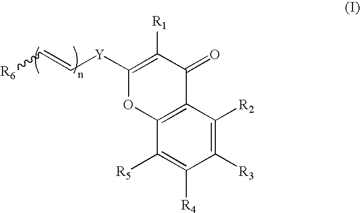

wherein R₁ is hydrogen, ORₐ or substituted or unsubstituted C₁-C₁₂ alkyl, and particularly preferred is a substituted or unsubstituted C₁-C₆ alkyl, methyl, ethyl, propyl, isopropyl and butyl are particularly preferred.

Particularly preferred R₂, R₃, R₄ and R₅ are hydrogen, ORₐ and OC(═O)Rₐ; wherein Rₐ has the same meaning given above. More preferred Rₐ is hydrogen and substituted or unsubstituted C₁-C₁₂ alkyl, even more preferred Rₐ is hydrogen and substituted or unsubstituted C₁-C₆ alkyl, and hydrogen, methyl, ethyl, propyl and isopropyl are the most preferred.

Particularly preferred X is O, S(O)ₘ or NR; wherein m is preferably 0 and R is preferably hydrogen and substituted or unsubstituted C₁-C₁₂ alkyl, more preferably hydrogen and substituted or unsubstituted C₁-C₆ alkyl, and hydrogen, methyl, ethyl, propyl, isopropyl and butyl are the most preferred.

The most preferred X is O.

In a preferred embodiment Y is a substituted or unsubstituted C₃-C₈ alkylene chain. The Y group may comprise one or more substituents. Substituted 1,4-butylene, 1,5-pentylene and 1,6-hexylene are the most preferred. These groups may be substituted in one or more positions. The preferred substituents are C₁-C₁₂ alkyl and OR′, wherein the R′ is as defined above. In a more preferred embodiment substituents are C₁-C₆ alkyl, OH, alkoxy and C(═O)alkyl. Even in a most preferred embodiment substituents are methyl, OH and —OCH₃.

Particularly preferred n is from 2 to 6 and more preferably 2 or 3.

Particularly preferred R₆ is selected from substituted or unsubstituted C₁-C₁₂ alkyl, substituted or unsubstituted C₂-C₁₂ alkenyl and substituted or unsubstituted C₂-C₁₂ alkyl; more preferred R₆ is an substituted or unsubstituted C₁-C₆ alkyl and substituted or unsubstituted C₂-C₆ alkenyl; 1-methylbutyl and 1-methylpropenyl are the most preferred.

Particularly preferred compounds of the invention are the following:

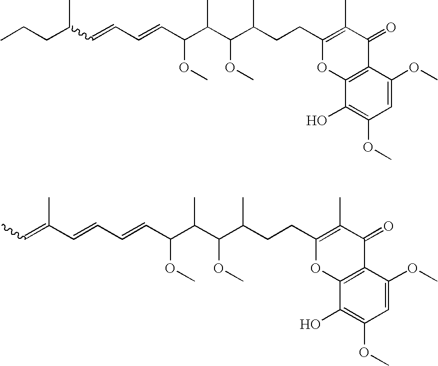

and their preferred stereochemistry is the following:

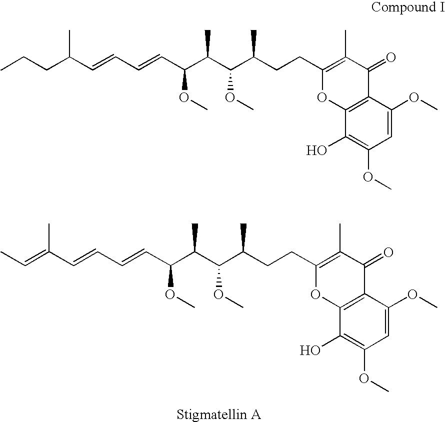

Compounds of the invention are readily made by synthetic methods. For example, compounds of this invention can be obtained with the procedures described in L. Domon et al. Tetrahedron Letters (2000), 41(29), 5501-5505; N. Adje et al. Tetrahedron Letters (2000), 41(29), 5495-5499; D. Enders et al. Chemistry European Journal (2000), 6(8), 1302-1309 or G. Hoefle et al. Liebigs Annalen der Chemie (1984), 12, 1883-1904. The synthetic routes can use combinations of steps taken from more than one of these articles.

In addition, some of the compounds of this invention can be of marine origin.

Compound I was isolated from a porifera, of the family Plakinidae, genus Corticium sp. A sample of the specimen was deposited in the “Instituto de Ciencias del Mar y Limnologia” of the Universidad Nacional Autónoma de México in Mazatlan, in Mexico and with the reference code LEB-ICML-UNAM-10-2004. This porifera was collected by hand using SCUBA diving in Wallis et Futuna (13° 22′ 36″ S, 176° 15′ 37″ W) at a depth ranging between 9 and 26 m, and its description is the following:

Family Plakinidae: Plakinidae Schulze, 1880 have encrusting growth forms. The body structure is simple, with the aquiferous system varying from simple asconoid construction to more complex folding and elaborate canal systems. The mineral skeleton consists of di-, tri- or tetractinal spicules, often with branched ends (lophotetractines); siliceous spicules and spongin fibres may be lacking in one genus, Oscarella, which has only collagenous fibrillar spongin in the mesohyl. Encrusting or massive growth forms; simple body structure with aquiferous system varying from simple asconoid construction to more complex folding and elaborate canal systems; mineral skeleton composed of relatively small calthrops and/or derivatives (diods or triods), often with branched ends (lophotetractines), generally arranged uniformly within sponge; spicules usually surround aquiferous system in regular “alveolar” arrangement; siliceous spicules and spongin fibres absent in one genus (Oscarella), having only collagenous fibrillar spongin in mesohyl; choanocyte chambers with 300-500 choanocytes, usually eurypylous, occasionally aphodal; larvae unique amphiblastula type.

Genus Corticium sp: Thinly encrusting, contractile surface; spiculation exclusively tetractines of single size and candelabras, although spicules occasionally absent completely; aphodal choanocyte chambers.

An important feature of the above described compounds of formula I is their bioactivity and in particular their cytotoxic and their inhibitory of EGFR intracellular signalling activity.

With this invention we provide novel pharmaceutical compositions of compounds of general formula I that possess cytotoxic and inhibitory of EGFR intracellular signalling activity, and their use as antitumor agents. Thus the present invention further provides pharmaceutical compositions comprising a compound of this invention, a pharmaceutically acceptable salts, derivatives, prodrugs or stereoisomers thereof with a pharmaceutically acceptable carrier.

Examples of pharmaceutical compositions include any solid (tablets, pills, capsules, granules etc.) or liquid (solutions, suspensions or emulsions) composition for oral, topical or parenteral administration.

Administration of the compounds or compositions of the present invention may be by any suitable method, such as intravenous infusion, oral preparations, and intraperitoneal and intravenous administration. We prefer that infusion times of up to 24 hours are used, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions containing compounds of the invention may be delivered by liposome or nanosphere encapsulation, in sustained release formulations or by other standard delivery means.

The correct dosage of the compounds will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The compounds and compositions of this invention may be used with other drugs to provide a combination therapy. The other drugs may form part of the same composition, or be provided as a separate composition for administration at the same time or at different time.

Antitumoral activities of these compounds include, but are not limited, lung cancer, colon cancer, breast cancer, cervix cancer, kidney cancer, leukemia, liver cancer, ovarian cancer, pancreas cancer, prostate cancer and stomach cancer.

### EXAMPLES

### Example 1

### Description of the Marine Organism and Collection Side

Corticium sp. was collected by hand using SCUBA diving in Wallis et Futuna (13° 22′ 36″ S, 176° 15′ 37″ W) at a depth ranging between 9 and 26 m. The material was identified by JoséLuis Carballo (Universidad Autónoma Nacional de Méjico). A sample of the specimen is deposited in the “Instituto de Ciencias del Mar y Limnologia” of the Universidad Nacional Autónoma de México in Mazatlan, Mexico. The reference code is: LEB-ICML-UNAM-10-2004.

### Example 2

### Isolation of Compound I

The frozen sponge of example 1 (38 g) was triturated and extracted with H₂O and a mixture of MeOH:CH₂Cl₂ (1:1) at room temperature. The organic extract was evaporated under reduced pressure to yield a crude of 0.22 g. This material was chromatographed (VLC) on Lichroprep RP-18 with a stepped gradient from H₂O to MeOH and subsequently MeOH:CH₂Cl₂ (1:1) and CH₂Cl₂. Fractions eluted with MeOH (23.3 mg) and MeOH:CH₂Cl₂ (1:1) (106.0 mg) were subjected to semipreparative reversed phase HPLC (X-Terra RP-18, 10×150 mm, isocratic H₂O:CH₃CN 40:60 for 5 min, then gradient to 80% CH₃CN in 15 min, UV detection) to yield 5.6 mg of Compound I as a colourless oil.

Compound I: colourless oil. ESIMS m/z; 531 [M+H]⁺, 499 [M+H-MeOH]⁺, 1083 [2M+Na]⁺.

¹H (500 MHz) and ¹³C NMR (125 MHz) see Table 1.

TABLE 1
¹H and ¹³C RMN data of Compound I (CD₃OD, 500 and 125 MHz).

| N^{o} | ¹H (Multiplicity, J) | ¹³C |
|---|---|---|
| 2 | — | 148.1 |
| 3 | — | 129.0 |
| 4 | — | 152.6 |
| 5 | 6.63 (s) | 94.0 |
| 6 | — | 153.9 |
| 7 | — | 108.3 |
| 8 | — | 179.7 |
| 9 | — | 117.3 |
| 10 | — | 165.5 |
| 11 | 2.84 (ddd, 14.5, 9.0, 5.5) | 30.5 |
| | 2.70 (ddd, 14.5, 8.0, 8.0) | |
| 12 | 1.92 (m) | 28.3 |
| | 1.55 (m) | |
| 13 | 1.74 (m) | 35.5 |
| 14 | 3.11 (dd, 9.0, 2.5) | 88.6 |
| 15 | 1.65 (ddq, 9.0, 2.5, 7.0) | 42.9 |
| 16 | 3.83 (dd, 7.5, 2.5) | 82.6 |
| 17 | 5.48 (dd, 15.0, 7.5) | 129.2 |
| 18 | 6.13 (dd, 15.0, 10.0) | 134.3 |
| 19 | 6.03 (dd, 15.0, 10.0) | 131.3 |
| 20 | 5.54 (dd, 15.0, 7.5) | 141.8 |
| 21 | 2.17 (m) | 37.8 |
| 22 | 1.29 (m), 2H | 40.4 |
| 23 | 1.29 (m), 2H | 21.5 |
| 24 | 0.89 (t, 7.0), 3H | 14.5 |
| 25 | 0.99 (d, 7.0), 3H | 21.0 |
| 26 | 0.74 (d, 7.0), 3H | 10.5 |
| 27 | 1.14 (d, 7.0), 3H | 18.2 |
| 28 | 1.98 (s), 3H | 10.0 |
| 29 | 3.88 (s), 3H | 56.7 |
| 30 | 3.99 (s), 3H | 56.9 |
| 31 | 3.46 (s), 3H | 61.6 |
| 32 | 3.21 (s), 3H | 56.5 |

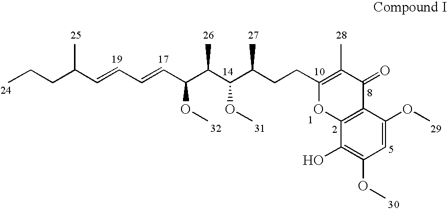

### Example 3

### Bioassays for Antitumor Screening

The finality of these assays was to interrupt the growth of a “in vitro” tumor cell culture by means of a continued exhibition of the cells to the sample to be testing.

### Cell Lines

| Name | N^{o} ATCC | Species | Tissue | Characteristics |
|---|---|---|---|---|
| A549 | CCL-185 | human | lung | lung carcinoma “NSCL” |
| HT29 | HTB-38 | human | colon | colon adenocarcinoma |
| MDA-MB-231 | HTB-26 | human | breast | breast adenocarcinoma |

A colorimetric type of assay, using sulforhodamine B (SRB) reaction has been adapted for a quantitative measurement of cell growth and viability [following the technique described by Philip Skehan et al. (1990), New colorimetric cytotoxicity assay for anticancer drug screening, J. Natl. Cancer Inst., 82:1107-1112].

This form of assay employed 96 well cell culture microplates of 9 mm diameter (Faircloth et al. Methods in cell science, (1988), 11(4), 201-205; Mosmann, Journal of. Immunological Methods (1983), 65(1-2), 55-63. Most of the cell lines were obtained from American Type Culture Collection (ATCC) derived from different human cancer types.

Cells were maintained in RPMI 1640 10% FBS, supplemented with 0.1 g/L penicillin and 0.1 g/L streptomycin sulphate and then incubated at 37° C., 5% CO₂ and 98% humidity. For the experiments, cells were harvested from subconfluent cultures using trypsin and resuspended in fresh medium before plating.

Cells were seeded in 96 well microtiter plates, at 5×10³ cells per well in aliquots of 195 μL medium, and they were allowed to attach to the plate surface by growing in drug free medium for 18 hours. Afterward, samples were added in aliquots of 5 μL in a ranging from 10 to 10⁻⁸ μg/mL, dissolved in DMSO:EtOH:PBS (0.5:0.5:99). After 48 hours exposure, the antitumor effect were measured by the SRB methodology: cells were fixed by adding 50 μL of cold 50% (wt/vol) trichloroacetic acid (TCA) and incubated for 60 minutes at 4° C. Plates were washed with deionised water and dried. One hundred μL of SRB solution (0.4% wt/vol in 1% acetic acid) was added to each microtiter well and incubated for 10 minutes at room temperature. Unbound SRB was removed by washing with 1% acetic acid. Plates were air dried and bound stain was solubilized with Tris buffer. Optical densities were read on an automated spectrophotometric plate reader at a single wavelength of 490 nm.

The values for mean +/−SD of data from triplicate wells were calculated. Some parameters for cellular responses could be calculated: GI=growth inhibition, TGI=total growth inhibition (cytostatic effect) and LC=cell killing (cytotoxic effect).

Compound I was obtained according to example 2 and Stigmatellin A (CAS Number: 91682-96-1) was purchased from Fluka (Ref.: 85865).

Table 2 illustrates data on the citotoxic activity of the compounds of the present invention.

TABLE 2
Activity Data (Molar)

| | | | Compound I | Stigmatellin A |
|---|---|---|---|---|
| Breast | MDA-MB-231 | GI₅₀ | 1.53E−7 | 3.89E−7 |
| | | TGI | 3.20E−6 | n.d. |
| | | LC₅₀ | n.d. | n.d. |
| Colon | HT29 | GI₅₀ | 8.67E−7 | 9.91E−7 |
| | | TGI | 5.84E−6 | n.d. |
| | | LC₅₀ | n.d. | n.d. |
| NSCL | A549 | GI₅₀ | 9.23E−8 | 6.02E−7 |
| | | TGI | 5.28E−7 | 1.94E−6 |
| | | LC₅₀ | 4.15E−6 | 7.58E−6 |

n.d. = not determined

### Cell Lines

| | Tumor |
|---|---|
| Name | Type |
| BT-474 | breast |
| RXF-393 | kidney |
| MOLT-4 | blood |
| Hep G2 | liver |
| ES-2 | ovarian |
| PANC-1 | pancreas |
| PC-3 | prostate |
| Hs 746T | stomach |

Cell lines were maintained in their respective growth media at 37° C., 5% CO₂ and 98% humidity. On the day before plating cells, cultures were refed with fresh, complete, antibiotic-free growth media. On the harvest (plating) day, cells were counted by Trypan Blue exclusion staining method, and seeded in 96 well microtiter plate in 190 μL of media and incubated for 24 h to allow cells to attach before addition of test drug. Plating was done by using Multidrop 384 Titan Device or multi-channel pipetter.

Stock solutions of Stigmatellin A (CAS Number: 91682-96-1, purchased from FLUKA (Ref: 85865)) were prepared in 100% DMSO at a concentration of 2 mg/mL. Stock solutions were considered to be stable for a period of 24 h only. Additional, serial dilutions, as described below, were prepared in serum-free media to achieve a final 20-fold treatment concentration. Ten μL of diluted test articles were added per well.

The cytotoxic effect was measured by the MTS Assay (Tetrazolium), which is a calorimetric method for determining the number of viable cells. After the 72 h of incubation with drug, 25 μL of MTS+PMS solution was added to each microtiter well and incubated for 4 hours at 37° C. Plates were then removed from incubator and placed on plate shaker for 5 minutes (covered with aluminium foil for protection from light). Optical densities were read at 490 nm on spectrophotometer plate reader. Data was analyzed using SoftMax program.

IC₅₀ was calculated (concentration at which 50% growth inhibition is measured). A regression curve using SoftMax program was generated, and then 50% inhibition concentration was manually interpolated and converted that concentration to molar (M) by dividing by the molecular weight of the compound.

Table 3 shows IC₅₀ (expressed as M) obtained for each cell line

TABLE 3
Antineoplastic in vitro activity of Stigmatellin A (Molar)

| Cell line | IC₅₀ (M) |
|---|---|
| BT-474 | 2.8 · E−6 |
| RXF-393 | 1.2 · E−5 |
| MOLT-4 | 3.1 · E−6 |
| Hep G2 | 9.8 · E−6 |
| ES-2 | 7.7 · E−6 |
| PANC-1 | 2.6 · E−5 |
| PC-3 | 1.4 · E−5 |
| Hs 746T | 2.1 · E−5 |

### Example 4

### EGFR Signalling Inhibition Assay Protocol

In this assay, the signal transduction pathway triggered by the activated Epidermal Growth Factor (EGF) membrane receptor is indirectly quantified using an EGF-responsive, API-mediated, luciferase reporter system.

HeLa-AP1, a subclone of HeLa cell line (human cervix carcinoma, ATCC# CCL-2) stably transfected with a construct containing the luciferase reporter gene under the control of the proximal promoter of the human collagenase-3 gene (consensus AP-1 response element TGACTCA at positions −56/−50) were used. Cells were maintained in DMEM supplemented with 10% FCS and 100 units/mL penicillin and streptomycin at 37° C. and 5% CO₂. HeLa-AP1 cells were pre-treated with the indicated compounds for 30 min before stimulation with EGF (25 ng/mL). After further 18 hours incubation, cell survival was estimated, for normalisation, by loading cells for 30 min with the vital fluorescent probe calcein-AM (0.5 μM). Fluorescence was quantified using a 1420 Victor² plate multilabel counter (Wallac). After that, cells were lysed and assayed for luciferase activity using the Bright-Glo system (Promega) and a 1450 Microbeta plate luminescence counter (Wallac-Trilux). Results were expressed as percentage of AP-1 activity inhibition as compared to control, untreated cells.

Compound I was obtained according to example 2 and Stigmatellin A (CAS Number: 91682-96-1) was purchased from FLUKA (Ref: 85865).

Table 4 illustrates data on the inhibition of EGFR intracellular signaling activity (AP-1 activity inhibition) of the compounds of the present invention.

TABLE 4
Activity Data (Molar)

| | IC₅₀ |
|---|---|
| Compound I | 2.26E−7 |
| Stigmatellin A | 9.72E−6 |

### Example 5

### Single-Administration Dose Range Finding in Mice

The finality of this assay was to determine the maximum tolerated dose (MTD) in mice by a single administration of the drug.

CD-1 male mice were used for this study, weighing ca. 25 g were randomly allocated to several dosing groups. Animals received a single intravenous administration of Stigmatellin A (CAS Number: 91682-96-1, purchased from Fluka (Ref: 85865)) dosed into the lateral vein of the tail. Once dosed, animals were observed for clinical signs at fixed intervals, up to 4 days after dosing. Mortality was recorded daily. The Maximum Tolerated Dose (MTD) was determined based on the mortality found in each dose level, calculated when mortality vs. dose is 0%.

Results and more specific details on the experimental protocol as well as the final results are summarized in Table 5:

TABLE 5
Maximum Tolerated Dose Data

| | Dose | | |
|---|---|---|---|
| | Levels | | |
| Animals/Groups | (mg/kg) | Vehicle | MTD (mg/kg) |
| 7M/7 | 16.0 | micelles | 0.44 |
| | 12.0 | | |
| | 8.0 | | |
| | 4.0 | | |
| | 2.0 | | |
| | 1.0 | | |
| | 0.5 | | |
| 6M/1 | 0.0 | | |

M = male

### Example 6

### Multiple-Administration Dose Range Finding in Mice

The finality of this assay was to determine the maximum tolerated multiple dose (MTMD) in mice by a multiple administration of the drug.

CD-1 male mice were used for this study, weighing ca. 25 g were randomly allocated to several dosing groups. Animals received a multiple doses by either intravenous or extravascular (intraperitoneal) route. Once dosed, animals were observed for clinical signs at fixed intervals, up to 4 days after dosing. Mortality was daily recorded. The MTMD was determined based on the mortality found in each dose level, calculated when mortality vs. dose is 0%.

Results for stigmatellin A (CAS Number: 91682-96-1, purchased from Fluka (Ref: 85865)) are summarized in Table 6:

TABLE 6
Maximum Tolerated Multiple Dose Data

| | Dose | | | |
|---|---|---|---|---|
| | Levels | | | (MTMD |
| Animals/Groups | (mg/kg) | Route/Schedule | Vehicle | mg/kg) |
| 5M/4 | 0.44 | iv/5DD | liposomes at | 0.14 |
| | 0.33 | | 0.096 mg/mL | |
| | 0.22 | | | |
| | 0.00 | | | |
| 5M/6 | 0.77 | ip/5DD | liposomes at | 0.28 |
| | 0.66 | | 0.035 mg/mL | |
| | 0.44 | | | |
| | 0.33 | | | |
| | 0.22 | | | |
| | 0.00 | | | |

M = male,
DD = daily dose.

1. A compound of formula (I) 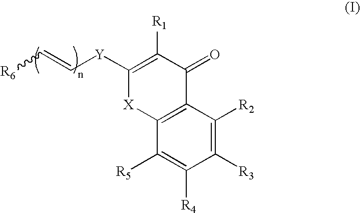
wherein R₁, R₂, R₃, R₄, R₅ and R₆ are each independently selected from the group consisting of hydrogen, ORₐ, OC(═O)Rₐ, halogen, substituted or unsubstituted C₁-C₁₂ alkyl, substituted or unsubstituted C₂-C₁₂ alkenyl and substituted or unsubstituted C₂-C₁₂ alkynyl;
wherein Rₐ is selected from the group consisting of hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl and substituted or unsubstituted heterocyclic group;
wherein X is O, S(O)ₘ or NR; wherein m is 0, 1 or 2;
wherein R is selected from the group consisting of hydrogen, substituted or unsubstituted C₁-C₁₂ alkyl, substituted or unsubstituted C₂-C₁₂ alkenyl and substituted or unsubstituted C₂-C₁₂ alkynyl;
wherein Y represents a substituted or unsubstituted C₁-C₁₂ alkylene chain;
wherein n is from 2 to 6;
and the wavy line ( [symbol] ) means that the bond can exist as (E)-isomer or (Z)-isomer;
or a pharmaceutically acceptable salt, derivative, prodrug or stereoisomer thereof;
with the proviso that
(a) when the structure is: 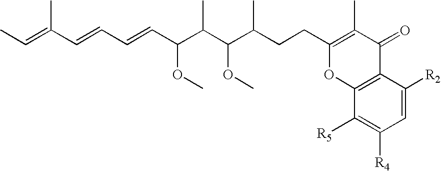
and R₂ is —OCH₃, R₄ is —OCH₃; R₅ is not —OH, —OCH₃, —OCOCH₃, —OCH₂CO₂H, —OCH₂Ph, or —OCH₂CO₂CH₂CH₃;
when R₂ is —OH, R₄ is —OCH₃; R₅ is not —OH or —OCH₃;
and
when R₄ is —OH, R₅ is —H; R₂ is not —OH or —OCH₃;
(b) when the structure is: 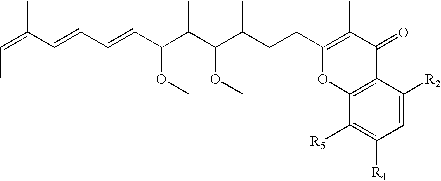
and R₂ is —OCH₃; R₄ is —OCH₃; R₅ is not —OH;
(c) when the structure is: 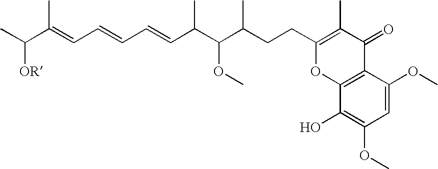
R′ is not H or methyl;
(d) when the structure is: 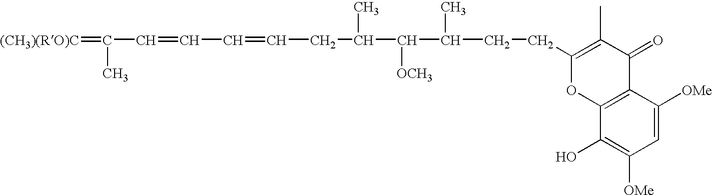
R′ is not methyl; and
(e) when the structure is: 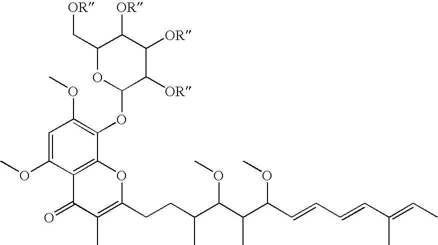
the R″ groups are not all —H or are not all —COCH₃.

2. A compound according to claim 1, wherein R₁ is hydrogen, ORₐ or substituted or unsubstituted C₁-C₁₂ alkyl, wherein Rₐ is as defined in claim 1.

3. A compound according to claim 1, wherein R₁ is a substituted or unsubstituted C₁-C₆ alkyl.

4. A compound according to any one of claim 3, wherein R₁ is selected from the group consisting of methyl, ethyl, propyl, isopropyl, and butyl.

5. A compound according to claim 1, wherein R₂, R₃, R₄ and R₅ are hydrogen, ORₐ or OC(═O)Rₐ.

6. A compound according to claim 5, wherein Rₐ is hydrogen or a substituted or unsubstituted C₁-C₁₂ alkyl.

7. A compound according to claim 6, wherein Rₐ is hydrogen or a substituted or unsubstituted C₁-C₆ alkyl.

8. A compound according to claim 7, wherein Rₐ is hydrogen, methyl, ethyl, propyl or isopropyl.

9. A compound according to claim 1, wherein X is O, S(O)ₘ or NR, m is 0 and R is hydrogen or a substituted or unsubstituted C₁-C₁₂ alkyl.

10. A compound according to claim 9, wherein R is hydrogen or a substituted or unsubstituted C₁-C₆ alkyl.

11. A compound according to claim 10, wherein R is hydrogen, methyl, propyl, isopropyl or butyl.

12. A compound according to claim 9, wherein X is O.

13. A compound according to claim 1, wherein Y is a substituted or unsubstituted C₃-C₈ alkylene chain.

14. A compound according to claim 13, wherein Y is substituted 1,4-butylene, 1,5-pentylene or 1,6-hexylene.

15. A compound according to claim 14, wherein 1,4-butylene, 1,5-pentylene or 1,6-hexylene are substituted in one or more positions with C₁-C₆ alkyl, OH, alkoxy or C(═O)alkyl.

16. A compound according to claim 1, wherein R₆ is substituted or unsubstituted C₁-C₁₂ alkyl, a substituted or unsubstituted C₂-C₁₂ alkenyl or a substituted or unsubstituted C₂-C₁₂ alkynyl.

17. A compound according to claim 16, wherein R₆ is a substituted or unsubstituted C₁-C₆ alkyl or a substituted or unsubstituted C₂-C₆ alkenyl.

18. A compound according to claim 17, wherein R₆ is 1-methylbutyl or 1-methylpropenyl.

19. A compound according to claim 1, wherein n is 2 or 3.

20. A compound according to claim 1, of formula:

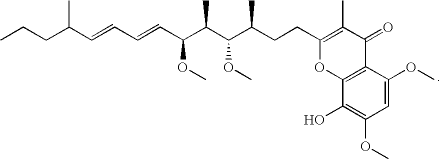

21. (canceled)

22. (canceled)

23. A pharmaceutical composition comprising a compound according to claim 1, or a pharmaceutically acceptable salt, derivative, prodrug or stereoisomer thereof, and a pharmaceutically acceptable diluent or carrier.

24. (canceled)

25. A method of treatment of cancer which comprises administering an effective amount of a compound as defined in claim 1, including those compounds excluded in the proviso of claim 1, or a pharmaceutically acceptable salt, derivative, prodrug or stereoisomer thereof.
